(19) United States
(12) Patent Application Publication
(10) Pub. No.: US 2023/0010922 A1
(43) Pub. Date: Jan. 12, 2023

(54) ORAL GUARD FOR LIGHT THERAPY

(71) Applicant: BioLight, Inc., Stevensville, MT (US)

(72) Inventor: Michael BELKOWSKI, Stevensville, MT (US)

(73) Assignee: BioLight, Inc., Stevensville, MT (US)

(21) Appl. No.: 17/810,657

(22) Filed: Jul. 5, 2022

Related U.S. Application Data
(60) Provisional application No. 63/218,699, filed on Jul. 6, 2021.

(51) Int. Cl. A61N 5/06

(52) U.S. Cl. CPC A61N 5/0603; A61N 2005/0606; A61N 2005/0626; A61N 2005/066; A61N 2005/0663; A61N 2005/0651

(57) ABSTRACT

Systems and techniques for configuring a light therapy oral guard include receiving sensed data from a first sensor associated with the light therapy oral guard, providing the sensed data from the first sensor to a machine learning model, receiving a machine learning output from the machine learning model based on the sensed data from the first sensor, the machine learning output comprising a light therapy oral guard configuration, and configuring the light therapy oral guard based on the light therapy oral guard configuration.

### CROSS-REFERENCE TO RELATED APPLICATIONS

This application claims the benefit of U.S. Provisional Patent Application 63/218,699, filed Jul. 6, 2021, the entire contents of which is incorporated herein by reference.

### TECHNICAL FIELD

Various embodiments of the present disclosure relate generally to an oral guard for light therapy and, more particularly, to devices configured to provide dual light emitting diode (LED) based red and near-infrared light therapy to the oral cavity of a user.

### BACKGROUND

Traditional mouth guards have been used to provide a buffer between an upper row of teeth and lower row of teeth to, e.g., prevent grinding of the two rows of teeth. Such guards are shaped to fit into a user's mouth such that the user's cheeks and lips surround an internal portion. Some mouth guards also may provide teeth whitening. However, there exists a need for providing oral health benefits to users that is not met by the existing mouth guards.

### SUMMARY OF THE DISCLOSURE

According to certain aspects of the disclosure, methods and systems are disclosed for using a light therapy oral guard.

Aspects disclosed herein include a method for configuring a light therapy oral guard, the method comprising: receiving sensed data from a first sensor associated with the light therapy oral guard; providing the sensed data from the first sensor to a machine learning model; receiving a machine learning output from the machine learning model based on the sensed data from the first sensor, the machine learning output comprising a light therapy oral guard configuration; and configuring the light therapy oral guard based on the light therapy oral guard configuration.

Aspects disclosed herein also include a light therapy oral guard comprising: an interior portion; an exterior portion, wherein at least a portion of the interior portion and the exterior portion is shaped to be positioned in an oral cavity; an exterior body portion comprising an input receptor, wherein the input receptor comprises a power control; one or more dual light emitting diodes (LEDs), wherein each dual LED is configured to output a first light having a first wavelength and a second light having a second wavelength.

Aspects disclosed herein also include a system for providing light therapy to an oral cavity, the system comprising: a light therapy oral guard comprising: an interior portion; an exterior portion, wherein the interior and exterior portions are configured to secure the light therapy oral guard to an oral cavity; one or more sensors attached to one or both of the interior portion or the exterior portion; one or more dual light emitting diodes (LEDs), wherein each dual LED is configured to output red light and near red light; at least one memory storing instructions; and at least one processor executing the instructions to perform a process, the processor configured to: receive sensed data sensed by the one or more sensors; receive a light therapy oral guard configuration based on the sensed data, the light therapy oral guard configuration comprising one or more of wavelengths of light, intensities of light, rates, durations, or frequencies for configuring the light therapy oral guard; and configure the light therapy oral guard based on the light therapy oral guard configuration.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIGS. 1A-1P depicts a plurality of views of a light therapy oral guard, according to one or more embodiments.

FIG. 2 depicts a diagram of a light therapy oral guard with exemplary dimensions, according to one or more embodiments.

FIGS. 3A and 3B also depict diagrams of a light therapy oral guard with exemplary dimensions, according to one or more embodiments.

FIG. 4 depicts a diagram of light distribution from a light therapy oral guard, according to one or more embodiments.

FIG. 5A depicts a diagram of light penetration from a red light emitted from a light therapy oral guard, according to one or more embodiments.

FIG. 5B depicts a diagram of light penetration from a near infrared light emitted from a light therapy oral guard, according to one or more embodiments.

FIGS. 6A and 6B depict sensors associated with a light therapy oral guard, according to one or more embodiments.

FIG. 7A depicts a flow chart for targeted light therapy, according to one or more embodiments.

FIG. 7B depicts a system environment for targeted light therapy, according to one or more embodiments.

FIG. 8 depicts a data flow for training a machine learning model, according to one or more embodiments.

FIG. 9 depicts an example system that may execute techniques presented herein

### DETAILED DESCRIPTION OF EMBODIMENTS

The terminology used herein may be interpreted in its broadest reasonable manner, even though it is being used in conjunction with a detailed description of certain specific examples of the present disclosure. Indeed, certain terms may even be emphasized herein; however, any terminology intended to be interpreted in any restricted manner will be overtly and specifically defined as such in this Detailed Description section. Both the general description and the detailed description are exemplary and explanatory only and are not restrictive of the features, as claimed.

As used herein, the terms “comprises,” “comprising,” “having,” including,” or other variations thereof, are intended to cover a non-exclusive inclusion such that a process, method, article, or apparatus that comprises a list of elements does not include only those elements, but may include other elements not expressly listed or inherent to such a process, method, article, or apparatus.

In this disclosure, relative terms, such as, for example, “about,” “substantially,” “generally,” and “approximately” are used to indicate a possible variation of ±10% in a stated value.

The term “exemplary” is used in the sense of “example” rather than “ideal.” As used herein, the singular forms “a,” “an,” and “the” include plural reference unless the context dictates otherwise.

As applied herein, an oral cavity may generally refer to a mouth and may include the areas including, approximate to, or in contact with the lips, the lining inside the cheeks and lips, the front two thirds of the tongue, the upper and lower gums, the floor of the mouth under the tongue, the roof of the mouth, and the area around the wisdom teeth.

According to implementations of the disclosed subject matter, a light therapy oral guard is provided for use in and/or around oral cavities. The light therapy oral guard may be configured to selectively provide red light (e.g., with a wavelength of approximately 630 nm, or within a range of approximately 600 nm-750 nm), near infrared light (e.g., with a wavelength of approximately 850 nm, or within a range of approximately 750-1000 nm), or a combination thereof. Generally, the light therapy oral guard may be configured to provide light within the range of approximately 600 nm-1000 nm.

The light therapy oral guard may be used to promote oral health and/or treat or mitigate oral health conditions such as inflammatory conditions, gingivitis, circulation, oral mycosis, or the like. The light therapy oral guard may also be used to expedite recovery (and reduce inflammation and/or pain) after oral surgery or other dental procedures. The light therapy oral guard, based on the use of both the red light and the near infrared light, may reduce inflammation, increase circulation, and optimize the functionality of mitochondria (e.g., to allow cells to generate energy more efficiently). As further discussed herein, the red light and/or near infrared light and/or a combination thereof may be used to treat surface conditions and/or sub-surface conditions (e.g., at a tissue level). As further discussed herein, the light therapy oral guard may be used to capture data related to biometric information, exhaled breath condensate (EBC), pH levels, saliva, chemicals, shapes, objects, food or food particles, electrical signals, tooth data, mitochondria, proteins, glucose, lactate, urea, serum, blood, and/or the like. The data may be captured using one or more sensors and may be used to configure the light therapy oral guard. The configuration may be to treat one or more conditions or to improve oral health.

The light therapy oral guard may include an internal portion, an external portion, and an external body portion. The internal portion and external portion may correspond to the portions that in whole or approximately in whole are placed inside a user's oral cavity during operation of the light therapy oral guard. The external body portion may correspond to the portion of the light therapy oral guard that in whole or approximately in whole remains outside a user's oral cavity during use of the light therapy oral guard.

The internal portion and/or external portion may be formed of any applicable material such as silicone or any other material that is anti-microbial and provides an amount of flexibility to comfortably fit inside a user's oral cavity. The external body portion be formed of any applicable material such as an Acrylonitrile Butadiene Styrene (ABS) material. The internal portion, the external portion, and/or the external body portion may be waterproof such that moisture does not come in contact with the electronic components of the light therapy oral guard.

The light therapy oral guard may include one or more input receptors which may be buttons, touch points (e.g., haptic response points), or the like, or may be voice, moisture (e.g., the light therapy oral guard may activate upon detecting moisture) or gesture activated. The input receptors may include a power button with a power control configured to provided power to the light therapy oral guard (e.g., on and off control), may include a timer, may include a setting adjustor, or the like. According to an implementation, two or more tasks may be performed by the same input receptor (e.g., power and timer operation may be conducted using the same button).

The light therapy oral guard may include a timer configured to automatically shut-off the light therapy oral guard after a given amount of time. The given amount of time may be pre-determined or dynamically determined. A pre-determined amount of time may be set during manufacture of the light therapy oral guard (e.g., may be 16 minutes, 10 minutes, etc.) and/or may be determined based on the intensity of the light (e.g., red light, infrared light) being emitted. For example, a user may set the timer while using the light therapy oral guard at a higher light intensity setting. A user may set the timer using an input receptor and/or via the GUI of an application used to control the light therapy oral guard. The timer may automatically be set for ten minutes based on the higher light intensity setting. At a different time, the user may set the timer while using the light therapy oral guard at a lower light intensity setting relative to the higher intensity setting. The timer may automatically be set for sixteen minutes based on the lower light intensity setting.

According to an implementation of the disclosed subject matter, the light therapy oral guard may include one or more sensors configured to capture data oral sensed data. Oral sensed data may include, but is not limited to, data related to biometric information, EBC, pH levels, saliva, chemicals, shapes, objects, food or food particles, electrical signals, tooth data, mitochondria, proteins, glucose, lactate, urea, serum, blood, and/or the like. The one or more sensors may be internal or external to the light therapy oral guard. The one or more sensors may be configured to detect a medical condition. The medical condition may be an oral condition that may be any applicable condition including, but not limited to, inflammatory conditions, gingivitis, circulation, oral mycosis, or the like. The one or more sensors may include a visual sensor, ambient condition detection sensor, pH sensor, biochemical sensor, or the like, as further discussed herein. The light therapy oral guard may be configured to adjust a setting based on the data obtained by the sensor. For example, the sensor may collect data and provide the data to a machine learning model. The machine learning model may be trained to output one or more settings of the light therapy oral guard based on the input sensor data. The output may be based on detecting a given oral condition or may be based on an oral condition known to or provided to the machine learning model. For example, the sensor data may sense sensed data (e.g., related to an oral condition) and the machine learning model may output one or more parameters to operate the light therapy oral guard based on the sensed data. The treatment may mitigate or treat a given oral condition. Alternatively, or in addition, the machine learning model may be a clinical decision support engine that may receive oral information from clinical guidelines, a user's dentist (e.g., via a server, network, or other connection to the user's dental data). Alternatively, or in addition, the machine learning model may generate a machine learning output based on cohort data, where the cohort data may indicate that a given output may cause the sensed data to improve to an improved condition.

The output may include a wavelength or set of wavelengths to output using the light therapy oral guard. The output may also include duration data such that the timer can be dynamically set to turn the light therapy oral guard off after the duration of time output by the machine learning model. Alternatively, the timer may be dynamically set to change a wavelength after a duration of time output by the machine learning model.

According to an implementation of the disclosed subject matter, settings for the light therapy oral guard may be adjusted based on user or automated input. As disclosed above, a machine learning model may output configurations (e.g., intensity, wavelength, duration, etc.) based on sensor data and/or data received at the machine learning model. One or more configurations of the light therapy oral guard may be adjusted based on user input. A user may provide the user input directly via input recipients on the light therapy oral guard. Alternatively, the light therapy oral guard may be connected to a user device (e.g., via a network, wired, or other wireless connection). The user may connect to the light therapy oral guard via an application (e.g., a mobile device application, a website or web application, a standalone controller, etc.) and provide setting input via a graphical user interface (GUI) of the application.

The light therapy oral guard may be powered using a battery. The battery may be charged in any applicable manner such as a Universal Serial Bus (USB) charger, wireless (e.g., Qi) charger, magnetic connection charger, or the like. The light therapy oral guard may include an indication of a low battery directly on the light therapy oral guard and/or may provide the indication via a user device (e.g., mobile phone) that the light therapy oral guard is connected to.

The light therapy oral guard may include a plurality of dual light emitting diodes (LEDs) across an internal portion. The dual LEDs may be or may include and may be manufactured or placed on a strip that is then inserted or placed on the internal portion of the light therapy oral guard. The dual LEDs may be equidistant from each other or may be spaced such that the outside edges (e.g., the portion of the light therapy oral guard that is closest to a user's wisdom teeth) have a higher distribution of dual LEDs and a central part (e.g., the portion of the light therapy oral guard that overlaps a user's middle teeth) has a lower distribution of chips, or vice versa. The dual LEDs may be arranged such that they face inside of a user's oral cavity (e.g., the dual LEDs face the side of a user's teeth and gums that face the external environment and/or the inner lips). As an example implementation, the light therapy oral guard may include sixteen dual LEDs spread equidistantly from each other.

The irradiance of the light therapy oral guard may be between approximately 5 mW/cm² and approximately 150 mW/cm². For example, the irradiance of the light therapy oral guard may be approximately 7 mW/cm². The irradiance may be variable based on one or more settings (e.g., as output by a machine learning model, set by a user, etc.).

The dual LEDs may be configured to output light in both the red wavelength and light in the near infrared wavelength. The same dual LEDs may include a lens that outputs red wavelength light, near infrared wavelength light, or a combination of the same. Accordingly, the light therapy oral guard may be configured to output either red light, infrared light, or a combination of the two using the same dual LEDs. In the example provided above including sixteen dual LEDs equidistant from each other, each of the sixteen dual LEDs may be configured to output red light, near infrared light, or a combination of the same.

According to implementations of the disclosed subject matter, the light therapy oral guard may emit less than 1 V/m at less than 0.5 inches away or less. For example, the light therapy oral guard may emit 0 V/m at less than 0.5 inches away. Such emission may be considered a safe amount of emission for use of the light therapy oral guard.

According to implementations of the disclosed subject matter, the dual LEDs of the light therapy oral guard may fluctuate at a frequency of 3 Hz or less. The fluctuation may be at a level that a human eye cannot perceive flickering of the dual LEDs of the light therapy oral guard.

According to implementations of the disclosed subject matter, the light therapy oral guard may incorporate security features. For example, a sensor may detect when the light therapy oral guard is placed inside an oral cavity (e.g., a pressure sensor to measure cheek/lip/teeth pressure, a moisture sensor, a biochemical sensor, etc.). Accordingly, the light therapy oral guard may activate only when the sensor determines that the light therapy oral guard is inside the oral cavity. Alternatively, or in addition, the light therapy oral guard may include a position or orientation sensor such that the light therapy oral guard activates only when the positon or orientation sensor detects that the light therapy oral guard is within a threshold position and/or orientation (e.g., where one or more orientation sensors detect a portion of an oral cavity is within 3 mm of a intended position). For example, the position or orientation sensor may deactivate the light therapy oral guard when it is removed from a user's oral cavity to prevent the red and/or near infrared light from being incident up on the user's eyes. The light therapy oral guard may also include a time used detection mechanism to detect how long a user has used the light therapy oral guard and/or at what intensity the light therapy oral guard was used for the duration. If the time used detection mechanism determines that the light therapy oral guard was used for a duration greater than a recommended duration and/or at an intensity greater than a recommended intensity for a given duration, the time used detection mechanism may automatically shut off the light therapy oral guard or reduce the intensity of the light therapy oral guard.

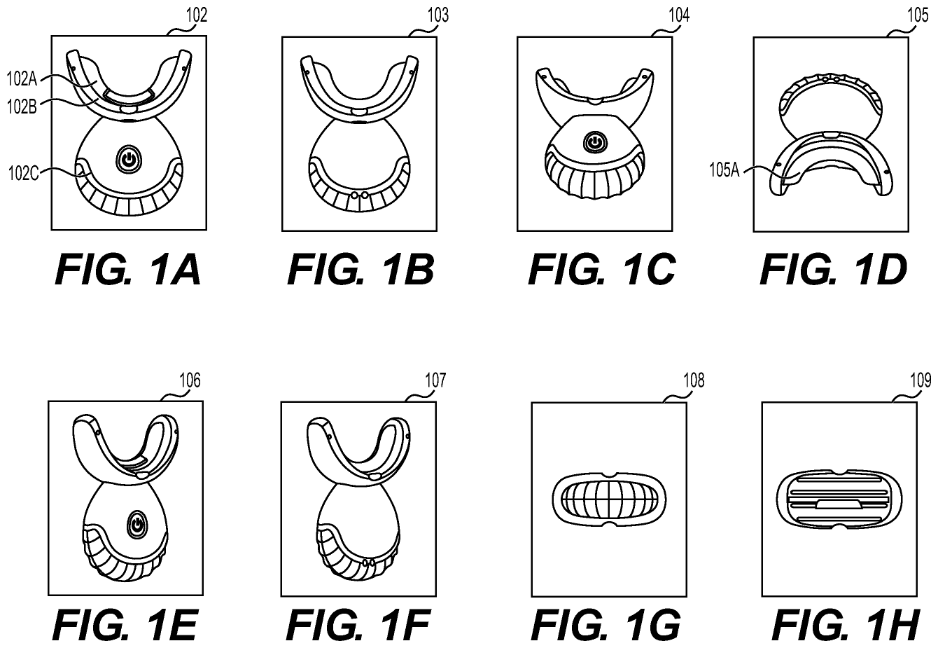

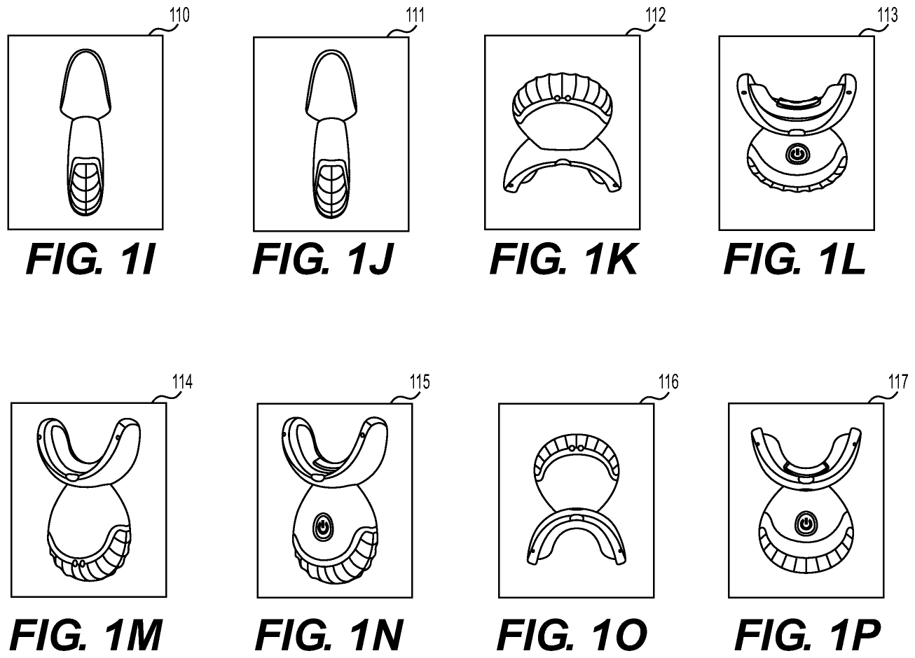

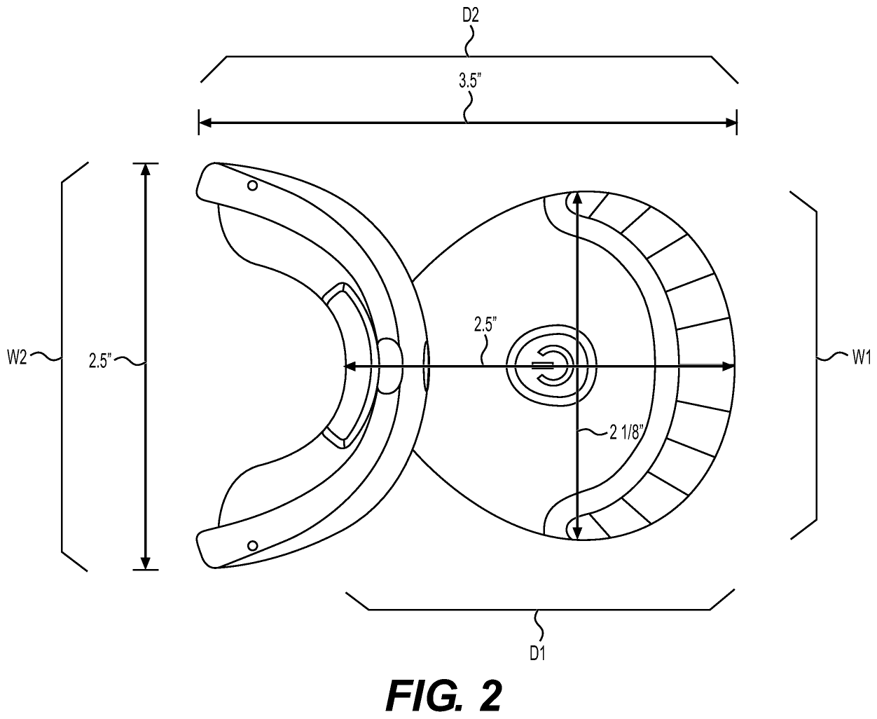

FIGS. 1A-1P provide a plurality of views 102 shown in FIG. 1A, 103 shown in FIG. 1B, 104 shown in FIG. 1C, 105 shown in FIG. 1D, 106 shown in FIG. 1E, 107 shown in FIG. 1F, 108 shown in FIG. 1G, 109 shown in FIG. 1H, 110 shown in FIG. 1I, 111 shown in FIG. 1J, 112 shown in FIG. 1K, 113 shown in FIG. 1L, 114 shown in FIG. 1M, 115 shown in FIG. 1N, 116 shown in FIG. 1O, and 117 shown in FIG. 1P of the light therapy oral guard in accordance with implementations of the disclosed subject matter. As shown, 102A corresponds to an interior portion of the light therapy oral guard, 102B corresponds to an exterior portion of the light therapy oral guard, and 102C corresponds to an exterior body portion of the light therapy oral guard. It will be understood that exterior portion 102B may be a side (e.g., a back side) of interior portion 102A. The interior portion 102A may include a curved section configured to align with the teeth and gums of a user. A teeth rest 105A may be provided in the middle of an upper and lower section of the interior portion 102A. The exterior body portion 1028 may include a textured area along a curved section along the back of the exterior body portion 1028, facing away from the interior portion 102A and exterior portion 1028. The textured area may include ribs, holes, or the like and may increase the grip-ability and/or reduce the risk of damage of the light therapy oral guard.

FIG. 2 shows a top view of an implementation of the light therapy oral guard. As shown, the light therapy oral guard may have a first depth D1 from the middle of the base of the exterior body portion facing away from the interior portion to an inner middle point of the interior portion facing away from the outer portion. D1 may be approximately 2.5 inches. The light therapy oral guard may have a second depth D2 from the base of the exterior body portion facing away from the interior portion to a far edge of the interior portion furthest away from the exterior body portion. D2 may be approximately 3.5 inches. The light therapy oral guard may have a first width W1 from first (e.g., right) side of the exterior body portion to a second (e.g., left) side of the exterior body portion, along the widest part of the exterior body portion in a first to second side direction. W1 may be approximately 2.125 inches. The light therapy oral guard may have a second width W2 from first (e.g., right) side of the interior portion to a second (e.g., left) side of the interior portion, across the widest part of the interior portion in a first to second side direction. W2 may be approximately 2.5 inches.

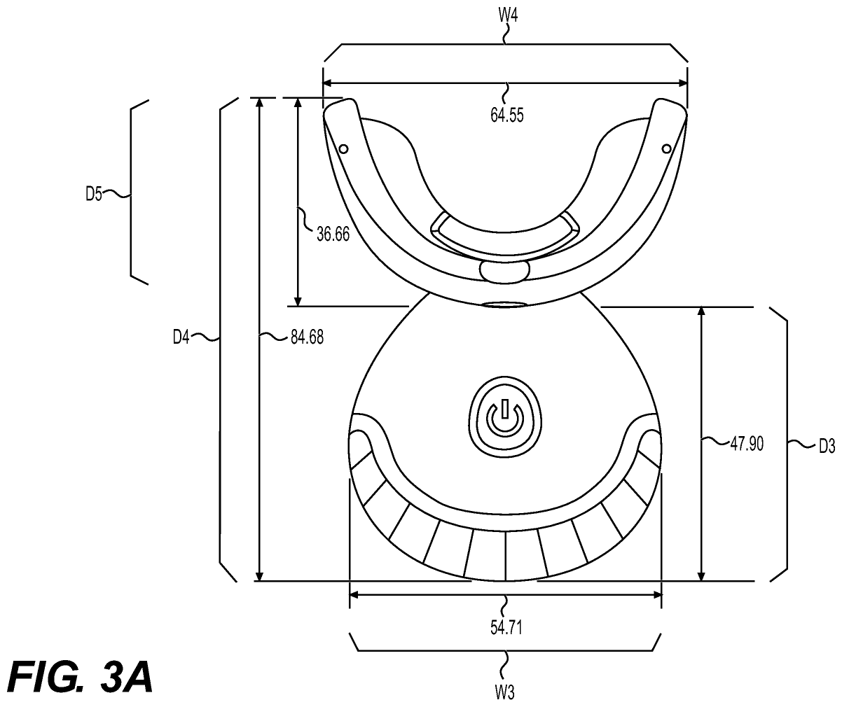

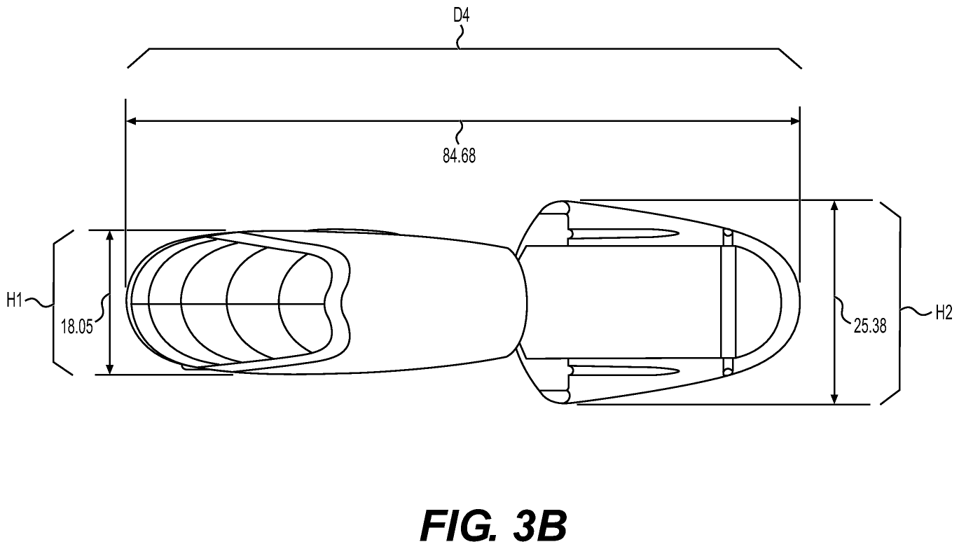

FIG. 3A shows another top view of an implementation of the light therapy oral guard. As shown, the light therapy oral guard may have a depth D3 from the base of the exterior body portion facing away from the interior portion to an upper most middle point of the exterior body portion facing the interior portion and exterior portion. D3 may be approximately 47.9 mm. The light therapy oral guard may have a depth D4 from the base of the exterior body portion facing away from the interior portion to a far edge of the exterior portion furthest away from the exterior body portion. D4 may be approximately 84.68 mm. The light therapy oral guard may have a depth D5 from the base of the exterior portion facing the exterior body portion to a far edge of the exterior portion furthest away from the exterior body portion. D5 may be approximately 36.66 mm. The light therapy oral guard may have a width W3 from first (e.g., right) side of the exterior body portion to a second (e.g., left) side of the exterior body portion, along the widest part of the exterior body portion in a first to second side direction. W3 may be approximately 54.71 mm. The light therapy oral guard may have a width W4 from first (e.g., right) side of the exterior portion to a second (e.g., left) side of the exterior portion, along the widest part of the exterior portion in a first to second side direction. W4 may be approximately 64.55 mm.

FIG. 3B shows a side view of an implementation of the light therapy oral guard. As shown, the light therapy oral guard may have a depth D4 from the base of the exterior body portion facing away from the interior portion to a far edge of the exterior portion furthest away from the exterior body portion, as also shown in FIG. 3A. D4 may be approximately 84.68 mm. The light therapy oral guard may have a height H1 from a first side (e.g., top) of the exterior body portion to a second side (e.g., bottom) of the exterior body portion. H1 may be approximately 18.05 mm. The light therapy oral guard may have a height H2 from a first side (e.g., top) of the exterior portion to a second side (e.g., bottom) of the exterior portion. H2 may be approximately 25.38 mm.

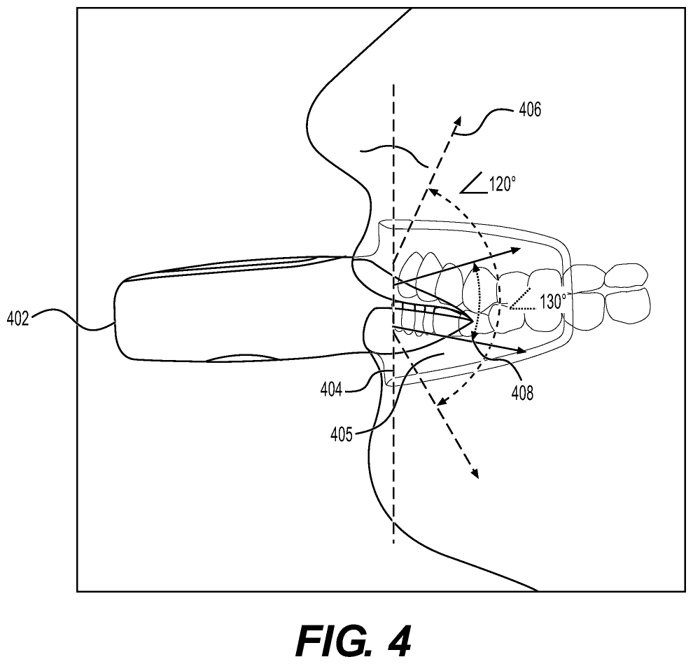

The light therapy oral guard may generate a beam angle of 120 degrees relative to the horizontal plane created through the middle of the light therapy oral guard, between a first (e.g., top) and second (e.g., bottom) side of the light therapy oral guard. FIG. 4 shows a diagram of a light therapy oral guard with an exterior body portion 402, exterior portion 404, and an interior portion 405 inside a user's oral cavity. As shown, the light therapy oral guard may emit light that has a 120 degree beam angle 406 such that the emitted light is incident upon the user's gums and respective upper and lower portions of the user's mouth. Accordingly, the light therapy oral guard may be able to treat a greater area of a user's oral cavity in comparison to a narrower beam device (e.g., with a beam angle 408 of 30 degrees that is only incident, at best, on a user's teeth).

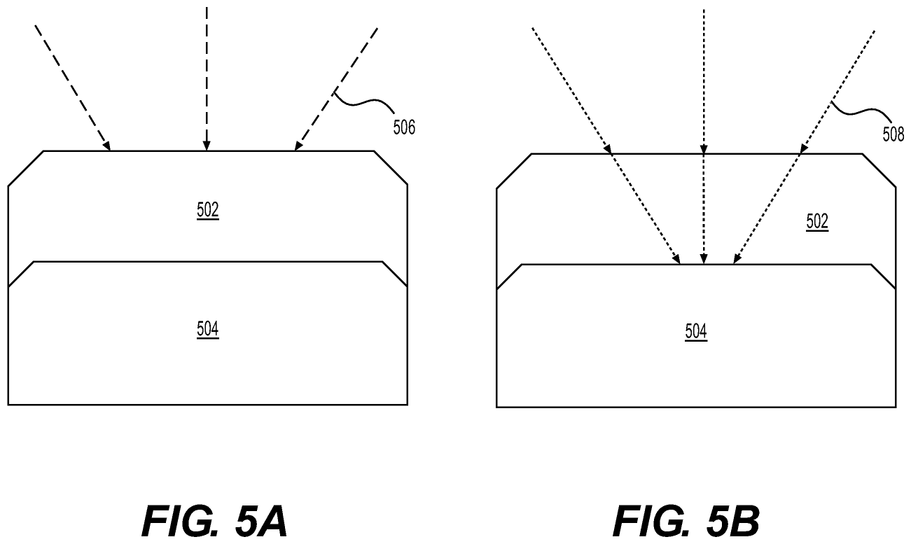

The dual LEDs disclosed herein may be each be configured to emit light (e.g., red light) that can interact with cells on a surface as well as light (e.g., near infrared light) that can interact with cells deeper than a surface. Such a configuration may have added health benefits in comparison to a configuration that only emits light incident on, for example, a surface level. FIGS. 5A and 5B depict diagrams representing an oral cavity's upper surface 502 (e.g., a gum surface) and a sub-surface area 504 (e.g., gum tissue). As disclosed herein, the light therapy oral guard may include dual LED circuits each configured to emit red light, near infrared light, and/or a combination of the two. The ability to emit red light, near infrared light, and/or a combination of the two may result in the light therapy oral guard efficiently reducing inflammation, increasing circulation, and/or optimizing functionality of mitochondria. As shown in FIG. 5A, red light 506 may emit from the light therapy oral guard and be incident on the upper surface 502. The red light 506 may interact with the upper surface 502 to reduce inflammation, increase circulation, and/or optimize functionality of mitochondria, or the like, at the upper surface 502. Additionally, as shown in FIG. 5B, near infrared light 508 may interact with the sub-surface area 504 to reduce inflammation, increase circulation, and/or optimize functionality of mitochondria, or the like, at the sub-surface area 504. Accordingly, using the dual LED circuits, the light therapy oral guard may have an effect on both the surface and sub-surface tissue that it interacts with.

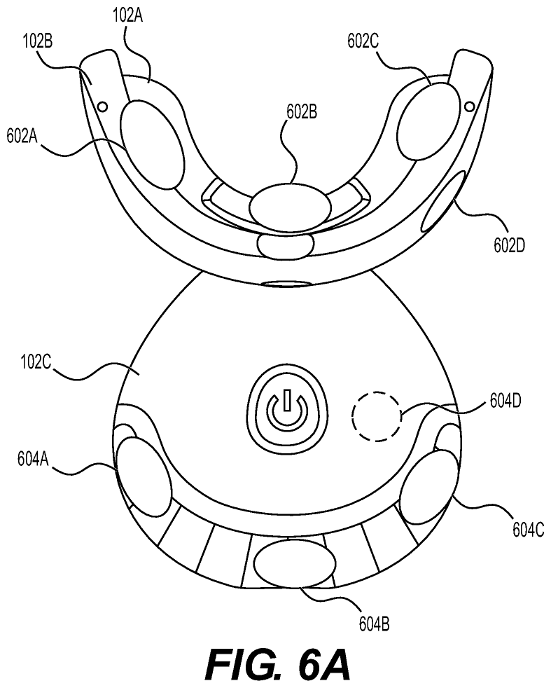

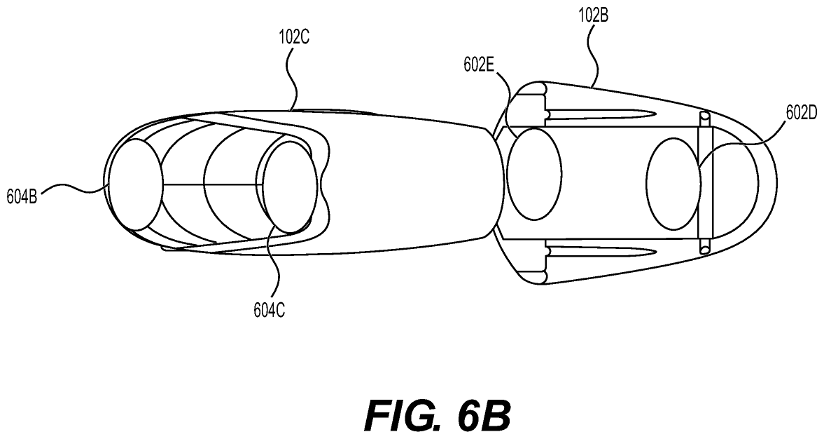

According to implementations of the disclosed subject matter, light therapy oral guard may include one or more sensors. FIGS. 6A and 6B depict sensors associated with a light therapy oral guard. As shown in FIG. 6A, sensors 602A, 602B, and/or 602C may be placed at or in the interior portion 102A of light therapy oral guard. Sensors 602A, 602B, and/or 602C may be attached to or contained within the interior portion 102A. Alternatively, as shown in FIG. 6A, sensors 602A, 602B, and/or 602C may be partially housed in or attached to exterior portion 102B such that they extend from exterior portion 102B through interior portion 102A. Sensor 602D may be positioned on the exterior portion 102B. As shown in FIG. 6A, sensors 604A, 604B, and/or 604C may be placed in or on the exterior body portion 102C of the light therapy oral guard. Sensor 604D may be placed inside the exterior body portion 102C. FIG. 6B also shows sensors 604B and 604C placed in or on the exterior body portion 102C of the light therapy oral guard. As shown in FIG. 6B, sensors 602E and/or 602D may be placed on the exterior portion 102B. Sensors 602E and/or 602D may be attached to exterior portion 102B, may extend into exterior portion 102B, or may extend into exterior portion 102B and may further extend into interior portion 102A (not shown).

It will be understood that the sensors shown in FIGS. 6A and 6B are example sensors and that one or more additional sensors may be associated with light therapy oral guard. Alternatively, not all the sensors shown in FIGS. 6A and 6B may be included in a given light therapy oral guard. Alternatively, or in addition, one or more sensors associated with light therapy oral guard may be external to the housing of the light therapy oral guard. Such sensors may communicate with light therapy oral guard or one or more processors associated with light therapy oral guard via a wired or wireless connection (e.g., a network connection, a Bluetooth®, connection, a near field connection, a line of sight connection, etc.). A given sensor, as those discussed herein, may be positioned, calibrated, and/or powered based on the sensing functionality of the respective sensor. For example, sensors configured to detect tissue properties may be positioned on the exterior portion 102B of the light therapy oral guard whereas sensors configured to detect saliva properties may be positioned on the interior portion 102A of the light therapy oral guard.

The sensors shown in FIGS. 6A and 6B or otherwise discussed herein may be a visual sensor, chips, laser sensor, temperature sensors, ambient condition detection sensor, pH sensor, biochemical sensor, motion sensors, material sensors, or the like. The sensors shown in FIGS. 6A and 6B or otherwise discussed herein may be configured to sense oral sensed data which may include, but is not limited to, data related to biometric information, EBC, pH levels, saliva, chemicals, shapes, objects, food or food particles, electrical signals, tooth data, mitochondria, proteins, glucose, lactate, urea, uric acid, microorganisms, serum, blood, and/or the like. The oral sensed data may be used to detect an oral condition or a treatment.

The sensors shown in FIGS. 6A and 6B or otherwise discussed herein may be powered using the same power source (e.g., a battery) as the length therapy oral guard. Alternatively, or in addition, the sensors may be powered using an external energy source (e.g., a power source, kinetic energy, heat energy, etc.).

The sensors shown in FIGS. 6A and 6B or otherwise discussed herein may be activated continuously or based on one or more triggers. A sensor activation may correspond to sensing of given sensed data by a given sensor. A sensor activation may include receiving an input (e.g., a physical input, a biochemical input, an electrical input, a motion input, etc.) and/or may include converting an input to a sensed signal. The sensed signal may be an electrical signal, a change of a state, a change of a property, or the like. For example, a sensor may be configured to detect pH levels. The sensor may continuously sense pH levels such that a pH detection mechanism constantly receives a sample (e.g., a saliva sample), applies the sample to a pH detection strip, and generates a signal (e.g., a reading) based on a physical property change of the strip.

A triggered based sensor activation may be based on a temporal trigger (e.g., based on a time, a duration of time, a chronic time, etc.), or an event based trigger. An event based trigger may be a trigger that is activated upon the occurrence of a given event. An event based trigger may be based on a signal from a sensor. For example, a position sensor or group of sensors may determine that the light therapy oral guard is in a user's mouth based on meeting one or more pressure criteria. The pressure sensor or group of sensors may detect that their proximity to corresponding areas of an oral cavity meets one or more thresholds. Accordingly, the pressure sensor or group of sensors may emit a signal indicating that the light therapy oral guard is in a user's mouth. Based on the signal, one or more other sensors may be activated, where the event in this case is the pressure sensor or group of sensors emitting the signal indicating that the light therapy oral guard is in a user's mouth.

An event based trigger may be based on an output determined based on user information, sensed information, or the like. User information may be a user history, a current user state, or the like. A current user state may be received from one or more components such as external sensors, a database, or the like (e.g., a blood pressure device, a health care database, etc.). The user information and/or sensed information (e.g., sensed by one or more sensors associated with the light therapy oral guard) may be input at a machine learning model and the machine learning model may determine when to trigger a sensor activation based on the user information and/or sensed information.

According to an implementation of the disclosed subject matter, one or more sensors associated with a light therapy oral guard may sense biochemical markers in bio fluids, such as sweat, tears, saliva and/or interstitial fluid. Such sensors may be non-invasive and may include one or more electrochemical and/or optical biosensors. Data sensed using such sensors may be used to identify or determine information related biomarkers including metabolites, bacteria, and hormones. For example, saliva may be collected by or near a light therapy oral guard. The saliva may be in contact with a non-invasive electrochemical sensor configured to detect one or more biomarkers including bacteria from the saliva. Concentration of certain biochemical markers in saliva may be highly relevant to those in blood, as a result of exchange between salivary glands and blood. Accordingly, as further discussed herein, sensed data (e.g., sensed salvia) may be used to determine or predict blood properties.

The non-invasive electrochemical sensor may sense electrochemical attributes of the saliva and may generate electrical signals based on the same. The electrical signals may be received at a processor located at or remote from the light therapy oral guard. The processor may convert the electrical signals to data that represents the presence of one or more biomarkers (e.g., a type, quantity, quality, etc., of bacteria). Alternatively, the non-invasive electrochemical sensor may itself be configured to output such data.

According to an implementation of the disclosed subject matter, one or more sensors associated with a light therapy oral guard may sense EBC content. EBC content may include, but is not limited to, mediators including adenosine, ammonia, hydrogen peroxide, isoprostanes, leukotrienes, nitrogen oxides, peptides and cytokines. Concentrations of these mediators are influenced by lung diseases and modulated by therapeutic interventions. Similarly, such one or more sensors may detect pH levels based on collected EBC. As further discussed herein, properties of EBC content and/or changes in the same may indicate the presence or probability of conditions (e.g., respiratory conditions).

According to an implementation of the disclosed subject matter, one or more sensors associated with a light therapy oral guard may sense volatile organic compounds (VOCs) biomarkers. VOC biomarkers may be indicative of environmental exposures such as that caused by particulate matter from burn pits, oil field fires, metal alloys, or the like.

According to an implementation of the disclosed subject matter, one or more sensors associated with a light therapy oral guard may be laser sensors. Such sensors may use one or more lasers to detect oral cavity properties. For example, laser sensors and the corresponding sensed data may be used for the early detection of decay. The laser sensors may be used to detect lesions (e.g., in early stages). A laser sensor may be placed on or near one or more teeth via the light therapy oral guard. The laser sensors may generate a digital readout, which may be used to determine tooth decay.

According to an implementation of the disclosed subject matter, one or more sensors associated with a light therapy oral guard may be configured to detect dental caries. Dental caries may be an infection resulting from tooth-adherent cariogenic bacteria, primarily Streptococcus Mutans, which metabolize sugars to produce acid, demineralizing the tooth structure over time. The sensors and/or a component of the light therapy oral guard may be configured to sense such bacteria, sugar, and/or acid and may include fluorescent components to facilitate detection of dental caries. The fluorescent components may include zinc oxide quantum dots/poly(dimethylsiloxane) (ZnO/PDMS) nanocomposite.

According to an implementation of the disclosed subject matter, one or more sensors associated with a light therapy oral guard may be pressure sensors, force sensors, or tooth movement sensors for orthodontic treatment monitoring. Such sensors may measure the force applied by orthodontic devices and/or any tooth changes based on the same. Such sensors may capture changes in tooth position over time. Such sensors may be implemented using a bracket or other component configured to detect force-moment (e.g., six force moment) of wires and/or brackets at one or more teeth. One or more stress sensors may be integrated on a chip via complementary metal oxide semiconductor (CMOS) technology. The chip may embedded into the light therapy oral guard. Based on the measured data, force-moment detection be determined. The data may be applied to or using one or more simulations. For example, isolated calibration loads may be complemented by using finite element (FE) simulations.

According to an implementation of the disclosed subject matter, one or more sensors associated with a light therapy oral guard may be temperature sensors. The temperature sensors may be used, for example, for monitoring peri-implant diseases. Temperature may be a targeting parameter of inflammation and the local temperature near a tooth or implant may be used as an indicator to monitor peri-implant diseases. Accordingly, a multi-channel temperature sensors may be microfabricated based on a photo-definable polyimide. Such sensors may output temperatures (e.g., over time) to detect temperature changes and/or peri-implant diseases.

According to an implementation of the disclosed subject matter, one or more sensors associated with a light therapy oral guard may be chemical sensors. Such sensors may sense food intake. Such sensors may include soft, low-profile, intraoral electronics configured for continuous real-time monitoring of sodium ingestion. Such sensors may include sodium ion-selective sodium electrodes (ISE), made of polymers with high selectivity, wide signal range, and rapid response time, selected for monitoring sodium levels in saliva.

According to an implementation of the disclosed subject matter, one or more sensors associated with a light therapy oral guard may be food physical sensors. Physical sensors may capture the motion of oral activity (e.g., over night while a user wears the light therapy oral guard). The oral activity may be sensed by such sensors and may be provided to a processor, as discussed herein. The oral activity may be provided as an input to a machine learning model which may generate a machine learning output, as further discussed herein. The machine learning output may be based on comparing the oral activity to known activity associated with known conditions (e.g., grinding) to identify an oral condition.

According to an implementation of the disclosed subject matter, one or more sensors associated with a light therapy oral guard may be optical sensors. Such sensors may use light-based techniques to detect oral cavity properties such as saliva properties, chemical properties, blood or blood flow properties, or the like. Such sensors may use a light-based techniques to quantify magnetic fields produced by neurons firing in the brain and may be used instead of magnetic resonance imaging (MRI) machines to create similar imaging, eliminating. Additionally, according to an implementation, the light therapy oral guard may include components (e.g., copper, galvanized steel, aluminum, etc.) that provide expensive cooling or electromagnetic shielding required when undergoes an MRI scan.

According to an implementation of the disclosed subject matter, one or more sensors associated with a light therapy oral guard may be infra-red or red sensors. Such sensors may capture high signal-to-noise and high-resolution photoplethysmography (PPG) measurements from deep beneath the oral cavity (e.g., up to approximately 20 mm or up to approximately 10× deeper than green light) to extract biometric sensed data.

According to an implementation of the disclosed subject matter, one or more sensors associated with a light therapy oral guard may be ultraviolet (UV) sensors. The UV sensors may use UV light to generate sensed data. According to an implementation, a user may swirl a fluorescent solution in the user's mouth. The user may then insert the light therapy oral guard and the UV sensors may detect oral properties (e.g., plaque) based on reminisce of the fluorescent solution. It will be understood that the fluorescent solution may not be required for the UV sensors to detect oral properties, though such a solution may improve the detectability of the same.

According to an implementation of the disclosed subject matter, one or more sensors associated with a light therapy oral guard may be biosensors. Biosensors may be used to sense sensed data that allows assessment of health and disease states. The biosensors may generate signals based on oral fluids, cells, microorganisms, etc., as well as other compounds that may be found in or pass through the oral cavity.

According to an implementation of the disclosed subject matter, one or more sensors associated with a light therapy oral guard may be glucose sensors. Such sensors may provide continuous glucose monitoring (CGM) based on oral cavity properties such as saliva properties. Such sensors may be configured to detect blood glucose or sense markers indicative of blood glucose. Such sensors may be configured to detect ketones and/or ketone properties which may be used to determine glucose levels. According to an implementation, such sensors may sense blood (e.g., blood that may be present in the oral cavity after brushing or flossing) and may detect blood glucose based on the blood.

According to an implementation of the disclosed subject matter, one or more sensors associated with a light therapy oral guard may be mitochondrial sensors. Mitochondrial sensors may be configured for quantum sensing such as by using one or more quantum objects. A quantum object may be the unpaired electron associated with an nitrogen-vacancy (NV) center in diamond which can be exploited as an extraordinarily sensitive room temperature magnetometer, deployed for nanoscale temperature measurements. Such objects may be used to detect temperature changes, as discussed herein.

Mitochondrial sensors may include a PINK1 sensor. PINK1 is a serine/threonine protein kinase which localizes to mitochondrion and regulates its function and turnover by sensing when mitochondria are damaged. PINK1 may be used for mitochondrial health by facilitating fusion and fission, mitophagy, and mitochondrial transport pathways, which serve as a quality control system to remove dysfunctional or damaged mitochondrion from the cell. Accordingly, mitochondrial sensors may detect the presence, quality, and/or quantity of PINK1.

Mitochondrial sensors may be configured to detect fluorescence-based assays including measurements of mitochondrial calcium, superoxide, mitochondrial permeability transition, and membrane potential.

According to an implementation of the disclosed subject matter, one or more sensors associated with a light therapy oral guard may be used to mimic blood tests. Such an implementation may use one or more of short-wavelength infra-red sensors, semiconductor photonics and/or electrooptic sensors, laser printed graphene (LIG) based electrode biosensors, or the like. A short-wavelength infra-red sensor may be used to detect the amount of sugar in a user's blood. A semiconductor photonics and/or electrooptic sensor may be configured to detect levels of glucose, lactate, urea, serum albumin, and/or other substances in a user's blood. LIG sensors may combine high electrical conductivity of graphene with an ultra-easy fabrication procedure that simply requires a CO2 laser printer. LIG sensors may be implemented a high porosity and an interlocking design to enhance the biosensor's sensitivity. Data output by such sensors may be used to generate results similar to a blood test.

According to implementations of the disclosed subject matter, a light therapy oral guard may be configured to generate sensed data using one or more of the sensors disclosed herein. The sensed data may be processed by a processor (e.g., an internal or external processor). A machine learning model may be used to generate a machine learning output. The machine learning output may include an indication, signal, instruction, or the like to configure the light therapy oral guard to output light at a wavelength, intensity, rate, duration, and/or frequency. The configuration of the light therapy oral guard may be, for example, to treat or otherwise improve a condition indicated based on the sensed data.

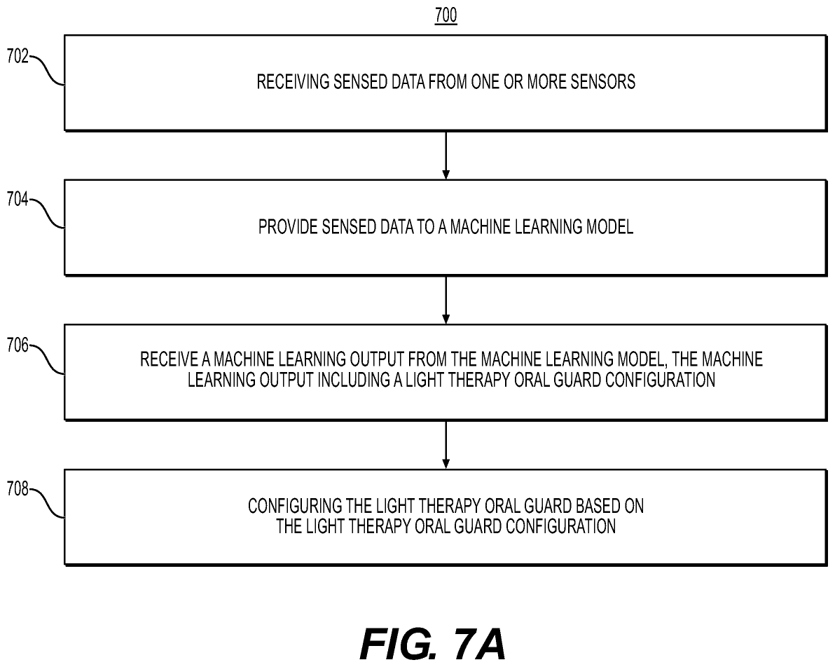

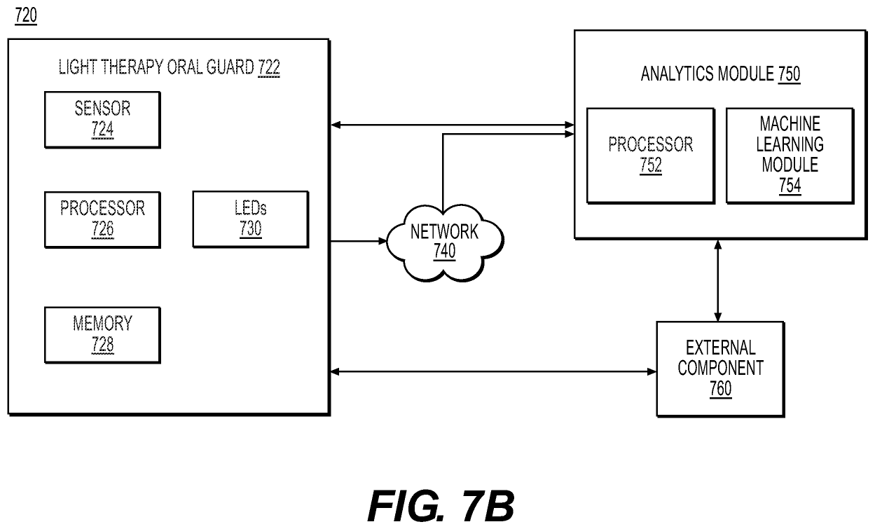

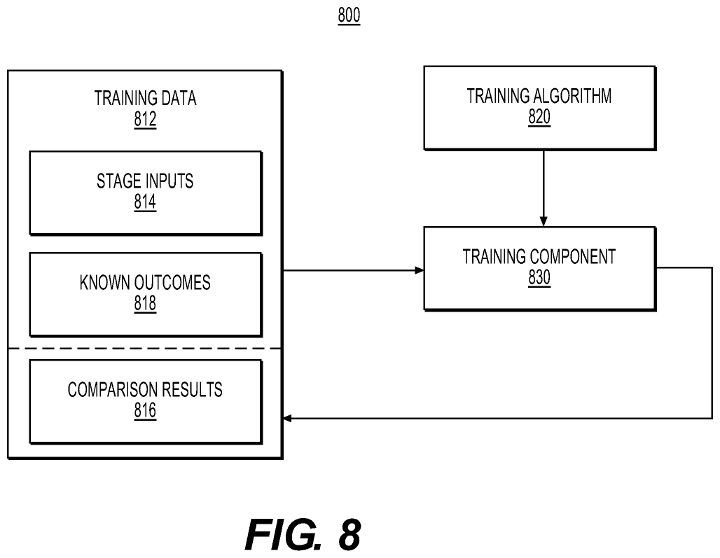

FIG. 7A depicts a flow chart 700 for targeted light therapy. At 702, sensed data from one or more sensors of the light therapy oral guard may be received. The sensed data may be any of the sensed data discussed herein generated by one or more of the sensors discussed herein. The sensed data may be generated by the one or more sensors continuously or based on an event, as discussed herein. The sensed data may be received an internal or external processor, as further discussed herein in reference to FIG. 7B. The sensed data may be sensed by one or more sensors in a first format (e.g., a sensor specific format, a signal, or the like) and may be converted into a second format (e.g., by a processor).

At 704, the sensed data may be provided to a machine learning model. The machine learning model may be trained based to generate a machine learning output based on sensed data. The machine learning model may be trained based on medical conditions, historical changes that effect medical conditions, light properties, or the like. The machine learning model may be trained by adjusting one or more of weights, layers, biases, nodes, or the like to correlate sensed data to medical conditions such that the correlation may be a probability or likelihood (e.g., above a respective threshold) that sensed data indicates the presence or likelihood of a medical condition. For example, the machine learning model may receive the sensed data and may apply the sensed data to one or more of weights, layers, biases, nodes, or the like to determine if the sensed data indicates the presence or likelihood of plaque. Alternatively, for example, the machine learning model may determine health properties of a user based on the sensed data. The health properties may be indicative of one or more medical conditions. The machine learning model may generate a machine learning output based on the correlation.

According to implementations of the disclosed subject matter, the machine learning model may be a single machine learning model or may include multiple machine learning models. For example, sensed data from different sensors may be provided to respective machine learning models and a central machine learning model configured to receive outputs from each of a plurality of respective machine learning models may generate the machine learning output.

According to implementations of the disclosed subject matter, the machine learning model may be trained and/or updated based on cohort data. Cohort data may correspond to sensed data and/or outcomes for a cohort of users. The cohort of users may be any group of users (e.g., other users that use similar light therapy oral guards, users with medical conditions, users that received light therapy for medical conditions, results of light therapy for users, or the like). For example, cohort data may include actual or simulated sensed data for the cohort of users. The cohort data may further include light therapy or other treatments implemented for the cohort of users and what effect the light therapy or other treatments had for the cohort of users. Light therapy treatments or other treatments correlated to light therapy treatments that had a positive effect for the cohort of users may be weighted heavily when training the machine learning model to generate a machine learning output. Accordingly, for example, sensed data for a given user may be compared to sensed data for the cohort of users. The machine learning model may apply greater weight to the layers, biases, weights, nodes, etc. trained based on the cohort of users that match the sensed data for a given user based on a matching threshold. Further, the machine learning model may apply greater weight to light therapy that had a positive effect for those matched cohort of users, when generating a machine learning output. Positive outcomes may include a reduction in presence or intensity of a given medical condition, the treatment of a medical condition, the prevention of a medical condition, or the like, for one or more medical conditions indicated by the sensed data.

At 706, a machine learning output may be received from the machine learning model. The machine learning output may include a light therapy oral guard configuration. Accordingly, the machine learning output may include a configuration that may be best indicated by the machine learning model to treat, mitigate, and/or prevent a medical condition for a given user.

A light therapy oral guard configuration may include one or more parameters of wavelengths of light, intensity of light, rate (e.g., pulse rate), duration of treatment, frequency of treatment, or the like. The wavelength(s) of light may correspond to the wavelengths that the light therapy oral guard is configured to output based on one or more other parameters. The machine learning output configuration may indicate how the light therapy oral guard outputs one or different wavelengths at different intensities, a given rate or different rates of output of the wavelengths, a duration or durations of time for output of the wavelengths, frequencies of output of the wavelengths, or the like. For example, the configuration may indicate that a first wavelength should be output at a first intensity for five minutes, at a second intensity for seven minutes. The configuration may further indicate that after the twelve total minutes of outputting the first wavelength, a four minute break where no wavelength is output should be implemented. The configuration may further indicate that after the break, a second wavelength should be output at a third intensity for two minutes, at a second intensity for nine minutes. The configuration may further indicate that the previous steps should be cycled through four times before the light therapy oral guard automatically shuts off.

According to an implementation, sensor data form a first sensor or group of sensors may be used to generate a machine learning output. Subsequently, sensed data from a second sensor or group of sensors may be used to update the machine learning output. The sensed data from the second sensor or group of sensors may be generated based on a first machine learning output configuration indicating a request for sensor data from the second sensor or group of sensors. For example, the machine learning model may determine that sensed data from a first sensor or group of sensors is not sufficient (e.g., in quantity, quality, type of data, etc.). Accordingly, the machine learning output may indicate a request for additional data from the second sensor or group of sensors. According to an implementation, the second sensor or group of sensors may include the first sensor or group of sensors (e.g., if additional data from the same sensors is requested).

At 708, the light therapy oral guard may be configured based on the light therapy oral guard configuration indicated the machine learning output. The light therapy oral guard may be configured using a processor, as further discussed herein. The configuration indicated by the machine learning output may be implemented until an updated configuration is received, or until the light therapy oral guard is reset using a reset signal (e.g., provided by a processor or via user input).

The intensity of light output by the light therapy oral guard may be adjusted by adjusting the power provided to one or more LEDs. Alternatively, or in addition, the intensity may be adjusted by a signal configured to increase or decrease the intensity output by one or more LEDs.

The wavelength output by the light therapy oral guard may be adjusted by activating and/or deactivating one or more LEDs. Alternatively, or in addition, the wavelength may be adjusted by modifying a property of the one or more LEDs. For example, each LED may have an on-board chip configured to modify the wavelength output by a given LED. A dual LED may include multiple bulbs configured to output one or more wavelengths and a wavelength may be adjusted by activating respective bulbs by providing a single to the on-board chip.

According to an implementation of the disclosed subject matter, updated sensed data may be provided to the machine learning model. The machine learning model may generate an updated machine learning output based on the updated sensed data. The updated machine learning output may be adjusted based on a current configuration (e.g., a previous machine learning output). The updated machine learning output may be adjusted based on updated cohort data that may also be received at the machine learning model. Accordingly, the light therapy oral guard may be continuously configured, at least in part based on changes effected by light therapy from pervious configurations and/or changes that a given user undergoes (e.g., health changes, diet changes, medication changes, etc.).

According to an implementation, the machine learning model may receive external input. The external input may be from one or more sensors external to the light therapy oral guard and/or user data. User data may be user diet information, user medication information, user health information, user activity information, or the like. For example, user diet information may be input by a user or an automated system. The user diet information may be used to determine a light therapy oral guard configuration such that, for example, a change to a salty diet may require changing a light (e.g., wavelength) output by the light therapy oral guard.

According to an implementation, the machine learning output may include an external device configuration. For example, the machine learning model may detect a glucose level of a given user. The glucose level may indicate the requirement of additional insulin at a given time. Accordingly, the machine learning model output may include a configuration for an insulin delivery device and the output may be provided to the insulin delivery device. The insulin delivery device may adjust an insulin output based on the machine learning output.

FIG. 7B depicts a system environment 720 for targeted light therapy in accordance with the subject matter disclosed herein. A light therapy oral guard 722 may include one or more sensors 724, processor 726, memory 728, and dual LEDs 730. In some implementations, processors 726 may include one or more microprocessors, microchips, or application-specific integrated circuits. Memory 728 may include one or more types of random-access memory (RAM), read-only memory (ROM), and cache memory employed during execution of program instructions and may further include storage including one or more databases, cloud components, servers, or the like. The storage may include a computer-readable, non-volatile hardware storage device that stores information and program instructions. Processor 726 may use data buses to communicate with memory 728, sensors 724, and/or dual LEDs 730. Processor 726 and/or another component (e.g., a transmitter and/or receiver) associated with light therapy oral guard 722 may be configured to transmit and/or receive data (e.g., sensed data, one or more configurations, etc.)

Analytics module 750 may be housed at light therapy oral guard 722 or may be an external component, as shown in FIG. 7B. Analytics module 750 may include a processor 752 and a machine learning module 754. Machine learning module 754 may be a set of instructions, code, or the like that are implemented (e.g., compiled) using processor 752). An external analytics module 750 may communicate with light therapy oral guard 722 via wired or wireless connection. The wireless connection may be via a network 740 such that analytics module 750 may be a remote or cloud component.

External component 760 may be an external sensor or an external device configured to communicate with light therapy oral guard 722 and/or analytics module 750 via wired or wireless connection.

According to an implementation, the light therapy oral guard may include the interior portion 102A and exterior portion 102B, but may not include the exterior body portion 102C. In this implementation, one or more input receivers (e.g., a power button) may be located on the exterior portion 102B. Similarly, external body sensors (e.g., sensors 604A, 604B, 604C, and/or 604D of FIGS. 6A and 6B) may be included on or in the external portion 102B.

According to an implementation of the disclosed subject matter, the light therapy oral guard may include a locking mechanism. The locking mechanism may be configured to lock an upper part of an oral cavity to the interior portion 102A and/or exterior portion 102B and/or a lower part of the oral cavity to the interior portion 102A and/or exterior portion 1028. The upper part of the oral cavity may correspond to an upper surface of the oral cavity (e.g., proximate to a user's nose). The lower part of the oral cavity may correspond to a lower surface of the oral cavity (e.g., proximate to a user's chin).

The locking mechanism may automatically or manually lock the upper part and/or the lower part of the oral cavity to the light therapy oral guard and/or to each other (e.g., such that a user's mouth/jaw remains closed). The locking mechanism may be a suction component, a force connection, or any applicable connection configured to attach the upper part to the lower part. The locking mechanism maybe configured to automatically lock the upper part and/or the lower part of the oral cavity based on predetermined criteria or based on a machine learning output configuration. For example, the locking mechanism may lock the upper part with the lower part based on sensor data indicating a grinding action by a user. For example, FIG. 3A shows a suction component 302 that may create a suction force between the light therapy oral guard and a portion of an oral cavity.

According to an implementation, the light therapy disclosed herein may be implemented using a toothbrush. The toothbrush may include one or more dual LEDs disclosed herein that may be activated during a brushing operation using the toothbrush. According to an implementation, a light therapy oral guard may be attached to the toothbrush such that a user may brush her teeth using the toothbrush and may further use the light therapy oral guard as disclosed herein. The toothbrush and the one or more dual LEDs may be powered from a central battery or energy source.

One or more implementations disclosed herein include a machine learning model. For example, as disclosed herein, a machine learning model may output operational parameters or settings to operate the light therapy oral guard based on, for example sensor data regarding oral health. A machine learning model disclosed herein may be trained using the data flow 800 of FIG. 8. As shown in FIG. 8, training data 812 may include one or more of stage inputs 814 and known outcomes 818 related to a machine learning model to be trained. The stage inputs 814 may be from any applicable source including sensor data. The known outcomes 818 may be included for machine learning models generated based on supervised or semi-supervised training. An unsupervised machine learning model may not be trained using known outcomes 818. Known outcomes 818 may include known or desired outputs for future inputs similar to or in the same category as stage inputs 814 that do not have corresponding known outputs.

The training data 812 and a training algorithm 820 may be provided to a training component 830 that may apply the training data 812 to the training algorithm 820 to generate a machine learning model. According to an implementation, the training component 830 may be provided comparison results 816 that compare a previous output of the corresponding machine learning model to apply the previous result to re-train the machine learning model. The comparison results 816 may be used by the training component 830 to update the corresponding machine learning model. The training algorithm 820 may utilize machine learning networks and/or models including, but not limited to a deep learning network such as Deep Neural Networks (DNN), Convolutional Neural Networks (CNN), Fully Convolutional Networks (FCN) and Recurrent Neural Networks (RCN), probabilistic models such as Bayesian Networks and Graphical Models, and/or discriminative models such as Decision Forests and maximum margin methods, or the like.

In general, any process or operation discussed in this disclosure that is understood to be computer-implementable, such as communicating with an application via a GUI, adjusting light therapy oral guard parameters or settings, etc., may be performed by one or more processors of a computer system. A process or process step performed by one or more processors may also be referred to as an operation. The one or more processors may be configured to perform such processes by having access to instructions (e.g., software or computer-readable code) that, when executed by the one or more processors, cause the one or more processors to perform the processes. The instructions may be stored in a memory of the computer system. A processor may be a central processing unit (CPU), a graphics processing unit (GPU), or any suitable types of processing unit.

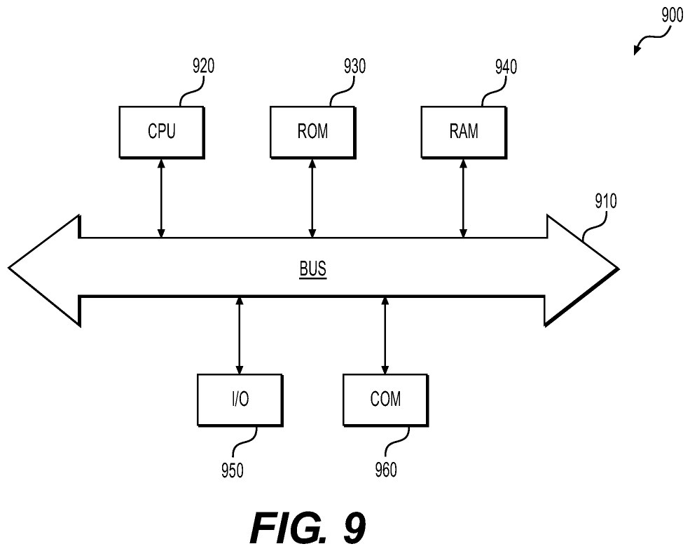

FIG. 9 depicts an example system 900 that may execute techniques presented herein. FIG. 9 is a simplified functional block diagram of a computer that may be configured to execute techniques described herein, according to exemplary embodiments of the present disclosure. Specifically, the computer (or “platform” as it may not be a single physical computer infrastructure) may include a data communication interface 960 for packet data communication. The platform may also include a central processing unit (“CPU”) 920, in the form of one or more processors, for executing program instructions. The platform may include an internal communication bus 910, and the platform may also include a program storage and/or a data storage for various data files to be processed and/or communicated by the platform such as ROM 930 and RAM 940, although the system 900 may receive programming and data via network communications. The system 900 also may include input and output ports 950 to connect with input and output devices such as keyboards, mice, touchscreens, monitors, displays, etc. Of course, the various system functions may be implemented in a distributed fashion on a number of similar platforms, to distribute the processing load. Alternatively, the systems may be implemented by appropriate programming of one computer hardware platform.

The general discussion of this disclosure provides a brief, general description of a suitable computing environment in which the present disclosure may be implemented. In one embodiment, any of the disclosed systems, methods, and/or graphical user interfaces may be executed by or implemented by a computing system consistent with or similar to that depicted and/or explained in this disclosure. Although not required, aspects of the present disclosure are described in the context of computer-executable instructions, such as routines executed by a data processing device, e.g., a server computer, wireless device, and/or personal computer. Those skilled in the relevant art will appreciate that aspects of the present disclosure can be practiced with other communications, data processing, or computer system configurations, including: Internet appliances, hand-held devices (including personal digital assistants (“PDAs”)), wearable computers, all manner of cellular or mobile phones (including Voice over IP (“VoIP”) phones), dumb terminals, media players, gaming devices, virtual reality devices, multi-processor systems, microprocessor-based or programmable consumer electronics, set-top boxes, network PCs, mini-computers, mainframe computers, and the like. Indeed, the terms “computer,” “server,” and the like, are generally used interchangeably herein, and refer to any of the above devices and systems, as well as any data processor.

Aspects of the present disclosure may be embodied in a special purpose computer and/or data processor that is specifically programmed, configured, and/or constructed to perform one or more of the computer-executable instructions explained in detail herein. While aspects of the present disclosure, such as certain functions, are described as being performed exclusively on a single device, the present disclosure may also be practiced in distributed environments where functions or modules are shared among disparate processing devices, which are linked through a communications network, such as a Local Area Network (“LAN”), Wide Area Network (“WAN”), and/or the Internet. Similarly, techniques presented herein as involving multiple devices may be implemented in a single device. In a distributed computing environment, program modules may be located in both local and/or remote memory storage devices.

Aspects of the present disclosure may be stored and/or distributed on non-transitory computer-readable media, including magnetically or optically readable computer discs, hard-wired or preprogrammed chips (e.g., EEPROM semiconductor chips), nanotechnology memory, biological memory, or other data storage media. Alternatively, computer implemented instructions, data structures, screen displays, and other data under aspects of the present disclosure may be distributed over the Internet and/or over other networks (including wireless networks), on a propagated signal on a propagation medium (e.g., an electromagnetic wave(s), a sound wave, etc.) over a period of time, and/or they may be provided on any analog or digital network (packet switched, circuit switched, or other scheme).

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method for configuring a light therapy oral guard, the method comprising:
receiving sensed data from a first sensor associated with the light therapy oral guard;
providing the sensed data from the first sensor to a machine learning model;
receiving a machine learning output from the machine learning model based on the sensed data from the first sensor, the machine learning output comprising a light therapy oral guard configuration; and
configuring the light therapy oral guard based on the light therapy oral guard configuration.

2. The method of claim 1, wherein the sensed data is one or more of biometric data, exhaled breath condensate (EBC) data, pH levels, saliva data, chemical data, shape data, object data, food data, electrical signal, tooth data, mitochondria data, protein data, glucose data, lactate data, urea data, serum data, blood data, light data, biochemical data, electrochemical data, volatile organic compounds (VOCs) biomarker data, laser data, force data, or movement data.

3. The method of claim 1, wherein the machine learning model is trained based on cohort data.

4. The method of claim 1, wherein the light therapy oral guard configuration comprises one or more of wavelengths of light, intensities of light, rates, durations, or frequencies for configuring the light therapy oral guard.

5. The method of claim 4, wherein the one or more wavelengths are selected from a range of approximately 600 nm-1000 nm.

6. The method of claim 1, further comprising:
receiving sensed data from a second sensor associated with the light therapy oral guard;
providing the sensed data from the second sensor to the machine learning model; and
receiving an updated machine learning output from the machine learning model based on the sensed data from the second sensor.

7. The method of claim 1, further comprising:
receiving updated sensed data from the first sensor after configuring the light therapy oral guard based on the light therapy oral guard configuration;
providing the updated sensed data from the first sensor to the machine learning model;
receiving an updated machine learning output from the machine learning model based on the updated sensed data from the first sensor, the updated machine learning output comprising an updated light therapy oral guard configuration; and
configuring the light therapy oral guard based on the updated light therapy oral guard configuration.

8. The method of claim 1, wherein the machine learning output further comprises an external component configuration.

9. The method of claim 8, further comprising outputting the external component configuration to an external component.

10. A light therapy oral guard comprising:
an interior portion;
an exterior portion, wherein at least a portion of the interior portion and the exterior portion is shaped to be positioned in an oral cavity;
an exterior body portion comprising an input receptor, wherein the input receptor comprises a power control;
one or more dual light emitting diodes (LEDs), wherein each dual LED is configured to output a first light having a first wavelength and a second light having a second wavelength.

11. The light therapy oral guard of claim 10, further comprising:
a processor, wherein the processor is configured to:
output sensed data sensed by the one or more sensors;
receive a light therapy oral guard configuration based on the sensed data, the light therapy oral guard configuration comprising one or more of wavelengths of light, intensities of light, rates, durations, or frequencies for configuring the light therapy oral guard; and
configure the light therapy oral guard based on the light therapy oral guard configuration.

12. The light therapy oral guard of claim 11, further comprising one or more sensors attached to one or both of the interior portion or the exterior portion, wherein the light therapy oral guard configuration is generated by a machine learning model based on the sensed data.

13. The light therapy oral guard of claim 10, wherein the light therapy oral guard is configured to generate a beam angle of approximately 120 degrees relative to a horizontal plane created through a middle of the light therapy oral guard, between an upper side and a lower side of the interior portion.

14. The light therapy oral guard of claim 10, wherein the first light is a red light, the first wavelength is approximately in a range of 600 nm-750 nm, the second light is a near red light, and the second wavelength is approximately in a range of 750 nm-1000 nm.

15. A system for providing light therapy to an oral cavity, the system comprising:
a light therapy oral guard comprising:
an interior portion;
an exterior portion, wherein the interior and exterior portions are configured to secure the light therapy oral guard to an oral cavity;
one or more sensors attached to one or both of the interior portion or the exterior portion;
one or more dual light emitting diodes (LEDs), wherein each dual LED is configured to output red light and near red light;
at least one memory storing instructions; and
at least one processor executing the instructions to perform a process, the processor configured to:
receive sensed data sensed by the one or more sensors;
receive a light therapy oral guard configuration based on the sensed data, the light therapy oral guard configuration comprising one or more of wavelengths of light, intensities of light, rates, durations, or frequencies for configuring the light therapy oral guard; and
configure the light therapy oral guard based on the light therapy oral guard configuration.

16. The system of claim 15, wherein the light therapy oral guard configuration is generated by a machine learning model based on the sensed data.

17. The system of claim 15, wherein the processor is further configured to:
apply the sensed data as an input to a machine learning model; and
receive a machine learning output from the machine learning model based on the sensed data, the machine learning output comprising the light therapy oral guard configuration.

18. The system of claim 15, wherein the processor is further configured to:
transmit the sensed data over a network; and
receive the light therapy oral guard configuration from the network.

19. The system of claim 15, further comprising an analytics module comprising a machine learning model configured to generate the light therapy oral guard configuration based on the sensed data.

20. The system of claim 15, further comprising an external component, wherein the processor is further configured to:
receive an external component configuration based on the sensed data; and
transmit the external configuration component to the external component.
